(19) United States
(12) Patent Application Publication
(10) Pub. No.: US 2026/0070919 A1
(43) Pub. Date: Mar. 12, 2026

(54) CRYSTAL FORM OF FIVE-MEMBERED AND SIX-MEMBERED HETEROCYCLIC COMPOUND, PREPARATION METHOD THEREFOR, AND USE THEREOF

(71) Applicant: BEIJING MAXINOVEL PHARMACEUTICALS CO., LTD., Beijing (CN)

(72) Inventors: Yuguang WANG, Guangdong (CN); Tianzhi WU, Guangdong (CN)

(21) Appl. No.: 19/108,917

(22) PCT Filed: Sep. 7, 2023
(86) PCT No.: PCT/CN2023/117439

§ 371 Date: Mar. 5, 2025

(30) Foreign Application Priority Data
Sep. 8, 2022 (CN) 202211100455.1

(51) Int. Cl. C07D 495/04; A61K 31/519

(52) U.S. Cl. CPC C07D 495/04; A61K 31/519

(57) ABSTRACT

Provided are a crystal form II or crystal form III of a compound represented by formula 1, a preparation method therefor, and use thereof. The crystal form has good physicochemical stability, does not easily absorb moisture, and has very important value for drug optimization and development.

The present application claims the right of the priority of Chinese patent application 2022111004551 filed on Sep. 8, 2022. The contents of the above Chinese patent application are incorporated herein by reference in their entirety.

### TECHNICAL FIELD

The present disclosure relates to a crystal form of a five-membered-fused six-membered heterocyclic compound, a preparation method therefor, and a use thereof.

### BACKGROUND

JAK-STAT signaling pathway, discovered in recent years, is a cytokine-stimulated signal transduction pathway involved in many important biological processes such as cell proliferation, differentiation, apoptosis, and immune regulation. Compared with other signaling pathways, the signaling pathway has a relatively simple transmission process, which mainly consists of three components, namely tyrosine kinase-related receptor, tyrosine kinase JAK, and transcription factor STAT.

JAK inhibitors can selectively inhibit JAK kinase, blocking the JAK/STAT pathway. Janus kinase is a non-receptor tyrosine protein kinase with four family members, namely JAK1, JAK2, TYK2, and JAK3. The first three are widely present in various tissues and cells, while JAK3 is only found in the bone marrow and lymphatic system. Clinically, JAK inhibitors are mainly used to screen therapeutic drugs for hematological diseases, tumors, rheumatoid arthritis, psoriasis, etc.

The JAK-STAT pathway is widely present in various tissues and cells of an organism, especially plays an important role in differentiation, proliferation, and anti-infection of lymphocyte cell lines, and is involved in the interaction of various inflammatory factors and signal transduction. The abnormal activation of the pathway is closely related to a variety of diseases. The search for and screening of JAK inhibitors contributes to the in-depth study of the regulatory mechanism of JAK-STAT, so as to provide new drugs and methods for the prevention and treatment of related diseases. In addition, the onset, growth, invasion, and metastasis of tumors are related to the JAK-STAT signal transduction pathway. The activation of STATs in normal signal transduction is rapid and transient, and the persistent activation of STATs is closely related to the malignant transformation of cells.

Recent studies have shown that rejection in organ transplantation, psoriasis, tissue and organ fibrosis, bronchial asthma, ischemic cardiomyopathy, heart failure, myocardial infarction, hematological diseases, and diseases of immune system are closely related to the JAK-STAT signal transduction pathway, which is not only important for the maintenance of normal physiological functions of cells, but also has an important regulatory effect on the onset and development of diseases.

WO2014/111037 discloses a JAK kinase inhibitor of a five-membered-fused six-membered heterocyclic compound, which has the structure of formula 1 as follows:

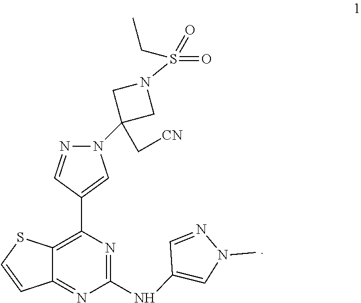 [SMILES: CCS(=O)(=O)N1CC(CC#N)(n2cc(-c3nc(Nc4cnn(C)c4)nc4ccsc34)cn2)C1]

It is well known that polymorphism is a common phenomenon in drug production. Different crystal forms exhibit significant differences in thermodynamics, kinetics, and physical properties, which may affect the stability of active pharmaceutical ingredients and formulations, the production process of formulations, dissolution, and bioavailability, and thus may affect the safety, efficacy, and quality controllability of drugs. Consequently, the study of polymorphism in drugs has always been the focus of the pharmaceutical industry. Therefore, the research and development of an advantageous crystal form with good fluidity, solubility, storage stability, and excellent bioavailability is crucial for the production, storage, and transportation of drugs.

### CONTENT OF THE PRESENT INVENTION

The technical problem addressed by the present disclosure is to improve the storage stability of a five-membered-fused six-membered heterocyclic compound represented by formula 1 in the prior art, thereby providing a crystal form of the five-membered-fused six-membered heterocyclic compound, a preparation method therefor, and a use thereof. The crystal form of the five-membered-fused six-membered heterocyclic compound of the present disclosure has good physicochemical stability, is not prone to moisture absorption, and is of great value for the optimization and development of drugs.

The present disclosure provides a crystal form II of the compound represented by formula 1, which has an X-ray powder diffraction pattern using CuKα radiation and represented by 2θ angles comprising characteristic peaks at 8.928°±0.2°, 10.781°±0.2°, 16.220°±0.2°, 16.817°±0.2°, 19.494°±0.2°, 19.955°±0.2°, and 25.026°±0.2°;

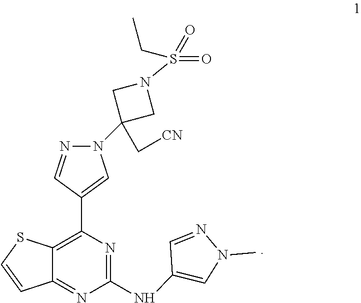 [SMILES: CCS(=O)(=O)N1CC(CC#N)(n2cc(-c3nc(Nc4cnn(C)c4)nc4ccsc34)cn2)C1]

(As shown above, the compound represented by formula 1 does not contain a solvent).

In some preferred embodiments of the present disclosure, the X-ray powder diffraction pattern for the crystal form II of the compound represented by formula 1, using CuKα radiation and represented by 2° angles, further comprises characteristic peaks at one or more of 6.976°±0.2°, 12.775°±0.2°, 13.316°±0.2°, 13.967°±0.2°, 16.450°±0.2°, 17.598°±0.2°, 19.377°±0.2°, 20.875°±0.2°, 22.119°±0.2°, 24.798°±0.2°, 25.752°±0.2°, 26.778°±0.2°, 27.959°±0.2°, 28.541°±0.2°, 28.957°±0.2°, 30.160°±0.2°, 33.201°±0.2°, 34.555°±0.2°, 34.916°±0.2°, 36.340°±0.2°, 36.602°±0.2°, and 37.986°±0.2°.

In some preferred embodiments of the present disclosure, the X-ray powder diffraction pattern for the crystal form II of the compound represented by formula 1 may also be substantially as shown in FIG. 5.

In some preferred embodiments of the present disclosure, the crystal form II of the compound represented by formula 1 has a differential scanning calorimetry pattern comprising an absorption peak at 205° C.±5° C., with a melting enthalpy preferably of 95.70 J/g.

In some preferred embodiments of the present disclosure, the differential scanning calorimetry pattern for the crystal form II of the compound represented by formula 1 may also be substantially as shown in FIG. 6.

In some preferred embodiments of the present disclosure, the crystal form II of the compound represented by formula 1 has a thermogravimetric analysis pattern with a weight loss of 0.01495% from 25° C. to the melting point, where the “%” represents weight percentage.

In some preferred embodiments of the present disclosure, the thermogravimetric analysis pattern for the crystal form II of the compound represented by formula 1 may also be substantially as shown in FIG. 7.

In some preferred embodiments of the present disclosure, the crystal form II of the compound represented by formula 1 has a dynamic vapor sorption pattern with a moisture absorption weight gain of 0.1047% in the range of 0% to 95% relative humidity, where the “%” represents weight percentage.

In some preferred embodiments of the present disclosure, the dynamic vapor sorption pattern for the crystal form II of the compound represented by formula 1 may also be substantially as shown in FIG. 8.

In some preferred embodiments of the present disclosure, the crystal form II of the compound represented by formula 1 has an infrared absorption spectrum determined by KBr pellet method comprising characteristic peaks at 3375 cm⁻¹, 3105 cm⁻¹, 2966 cm⁻¹, 2922 cm⁻¹, 1651 cm⁻¹, 1595 cm⁻¹, 1577 cm⁻¹, 1523 cm⁻¹, 1446 cm⁻¹, 1479 cm⁻¹, 1382 cm⁻¹, 1342 cm⁻¹, 1139 cm⁻¹, 1020 cm⁻¹, 883 cm⁻¹, and 686 cm⁻¹.

In some preferred embodiments of the present disclosure, the infrared absorption spectrum for the crystal form II of the compound represented by formula 1, determined by KBr pellet method, may also be substantially as shown in FIG. 9.

The present disclosure also provides a preparation method for the crystal form II of the compound represented by formula 1, comprising the following steps: slurrying a crystal form I of the compound represented by formula 1 in acetonitrile, followed by drying to obtain the crystal form II of the compound represented by formula 1; wherein the crystal form I of the compound represented by formula 1 has an X-ray powder diffraction pattern using CuKα radiation and represented by 2θ angles comprising characteristic peaks at 8.086°±0.2°, 11.879°±0.2°, 14.375°±0.2°, 15.434°±0.2°, 16.213°±0.2°, 17.372°±0.2°, 17.618°±0.2°, 19.066°±0.2°, 19.897°±0.2°, 22.997°±0.2°, 23.240°±0.2°, 24.033°±0.2°, 25.339°±0.2°, 25.641°±0.2°, 30.179°±0.2°, 31.164°±0.2θ, and 32.816°±0.2θ.

In the preparation method for the crystal form II, the volume-to-mass ratio of the acetonitrile to the compound represented by formula 1 is a conventional ratio in the art, preferably 10 to 30 times, for example, 20 times.

In the preparation method for the crystal form II, the slurrying is further followed by rotating at room temperature for equilibration.

In the preparation method for the crystal form II, the drying is natural drying.

The present disclosure also provides a preparation method for the crystal form II of the compound represented by formula 1, comprising the following steps: mixing the compound represented by formula 1 with acetone, followed by heating, dissolving, cooling at a low temperature, and drying to obtain the crystal form II of the compound represented by formula 1.

In the preparation method for the crystal form II, the mixing may be carried out at a rotational speed of 400 rpm.

In the preparation method for the crystal form II, the volume-to-mass ratio of the acetone to the compound represented by formula 1 is a conventional ratio in the art, preferably 20 to 40 times, for example, 30 times.

In the preparation method for the crystal form II, the heating is carried out in a water bath at a temperature of 40° C. to 60° C., for example, 50° C.

In the preparation method for the crystal form II, the dissolving is further followed by filtrating.

In the preparation method for the crystal form II, the cooling may be carried out at a temperature of −10° C. to −30° C. or less, for example, −20° C.

In the preparation method for the crystal form II, the cooling may be carried out for 10 hours to 16 hours, for example, 12 hours.

In the preparation method for the crystal form II, the drying may be natural drying.

The present disclosure also provides a preparation method for the crystal form II of the compound represented by formula 1, comprising the following steps:
(1) mixing the compound represented by formula 1 with a good solvent, followed by heating and dissolving;
(2) mixing the solution from step (1) with an anti-solvent under stirring, followed by drying to obtain the crystal form II of the compound represented by formula 1.

In the preparation method for the crystal form II, the good solvent may be one of ethyl acetate, acetone, dichloromethane, or tetrahydrofuran.

In the preparation method for the crystal form II, the heating may be carried out in a water bath at a temperature of 40° C. to 60° C., for example, 50° C.

In the preparation method for the crystal form II, the anti-solvent may be n-heptane or methyl tert-butyl ether.

In the preparation method for the crystal form II, the volume-to-mass ratio of the good solvent to the compound represented by formula 1 may be 30 to 180 times, for example, 30 times, 50 times, 90 times, or 180 times.

In the preparation method for the crystal form II, the volume ratio of the good solvent to the anti-solvent may be 1:(0.5 to 5), for example, 1:0.8, 1:1, 1:1.7, 1:2.9, 1:3.2, or 1:4.2.

In the preparation method for the crystal form II, in step (2), the mixing may be carried out by dropwise addition of the anti-solvent to the solution from step (1).

In the preparation method for the crystal form II, the drying may be natural drying.

The present disclosure also provides a crystal form III of the compound represented by formula 1, which has an X-ray powder diffraction pattern using CuKα radiation and represented by 2θ angles comprising characteristic peaks at 7.577°±0.2°, 10.415°±0.2°, 14.809°±0.2°, 19.797°±0.2°, 20.813°±0.2°, 21.939°±0.2°, 22.663°±0.2°, 27.689°±0.2°, and 29.791°±0.2°;

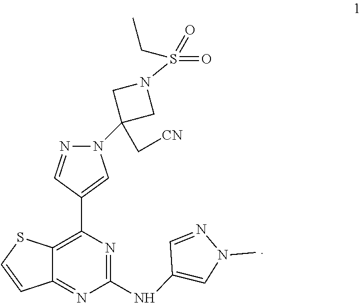 [SMILES: CCS(=O)(=O)N1CC(CC#N)(n2cc(-c3nc(Nc4cnn(C)c4)nc4ccsc34)cn2)C1]

(As shown above, the compound represented by formula 1 does not contain a solvent).

In some preferred embodiments of the present disclosure, the X-ray powder diffraction pattern for the crystal form III of the compound represented by formula 1, using CuKα radiation and represented by 2θ angles, further comprises diffraction peaks at one or more of 5.515°±0.2°, 8.137°±0.2°, 11.632°±0.2°, 16.258°±0.2°, 17.347°±0.2°, 19.318°±0.2°, 23.108°±0.2°, 24.664°±0.2°, 25.282°±0.2°, 25.998°±0.2°, 28.387°±0.2°, 30.347°±0.2°, 32.742°±0.2°, 34.932°±0.2°, 35.679°±0.2°, 37.573°±0.2°, and 38.218°±0.2°.

In some preferred embodiments of the present disclosure, the X-ray powder diffraction pattern for the crystal form III of the compound represented by formula 1 may also be substantially as shown in FIG. 10.

In some preferred embodiments of the present disclosure, the crystal form III of the compound represented by formula 1 has a differential scanning calorimetry pattern comprising an absorption peak at 162° C.±5° C., with a melting enthalpy preferably of 116.7 J/g.

In some preferred embodiments of the present disclosure, the differential scanning calorimetry pattern for the crystal form III of the compound represented by formula 1 may also be substantially as shown in FIG. 11.

In some preferred embodiments of the present disclosure, the crystal form III of the compound represented by formula 1 has a thermogravimetric analysis pattern with a weight loss of 0.1066% from 25° C. to the melting point, where the “%” represents weight percentage.

In some preferred embodiments of the present disclosure, the thermogravimetric analysis pattern for the crystal form III of the compound represented by formula 1 may also be substantially as shown in FIG. 12.

In some preferred embodiments of the present disclosure, the crystal form III of the compound represented by formula 1 has a dynamic vapor sorption pattern with a moisture absorption weight gain of 0.4084% in the range of 0% to 95% relative humidity, where the “%” represents weight percentage.

In some preferred embodiments of the present disclosure, the dynamic vapor sorption pattern for the crystal form III of the compound represented by formula 1 may also be substantially as shown in FIG. 13.

The present disclosure also provides a preparation method for the crystal form III of the compound represented by formula 1, comprising the following steps:
(1) mixing the crystal form I of the compound represented by formula 1 with a mixed solvent of methanol/water;
(2) mixing the solution from step (1) with water under stirring;
(3) drying to obtain the crystal form III of the compound represented by formula 1;
wherein the crystal form I of the compound represented by formula 1 has an X-ray powder diffraction pattern using CuKα radiation and represented by 2θ angles comprising characteristic peaks at 8.086°±0.2°, 11.879°±0.2°, 14.375°±0.2°, 15.434°±0.2°, 16.213°±0.2°, 17.372°±0.2°, 17.618°±0.2°, 19.066°±0.2°, 19.897°±0.2°, 22.997°±0.2°, 23.240°±0.2°, 24.033°±0.2°, 25.339°±0.2°, 25.641°±0.2°, 30.179°±0.2°, 31.164°±0.2°, and 32.816°±0.2°.

In the step (1), the volume ratio of the methanol to the water is preferably (5 to 15):1, for example, 10:1.

In the step (1), the volume-to-mass ratio of the mixed solvent to the compound represented by formula 1 is a conventional ratio in the art, preferably 90 to 110 times, for example, 100 times.

In the step (2), the volume-to-mass ratio of the water to the compound represented by formula 1 is a conventional ratio in the art, preferably 20 to 30 times, for example, 25 times.

In the step (2), the mixing is preferably carried out by dropwise addition of water to the solution from step (1).

In the step (2), the mixing is preferably carried out at a temperature of 45° C. to 55° C., for example, 50° C.

In the step (3), the drying is preferably vacuum drying.

In the step (3), the drying is preferably carried out at a temperature of 40° C. to 50° C., for example, 45° C.

The present disclosure also provides a use of the crystal form II or crystal form III of the compound represented by formula 1 in the manufacture of a JAK kinase inhibitor.

The present disclosure also provides a use of the crystal form II or crystal form III of the compound represented by formula 1 in the manufacture of a medicament for preventing and/or treating a disease related to JAK kinase.

In the present disclosure, the medicament may be used in combination with other therapeutic agents for preventing and/or treating a disease related to JAK kinase.

In the present disclosure, the other therapeutic agents may be used for preventing and/or treating a disease related to JAK kinase.

In the present disclosure, the disease related to JAK kinase includes, but is not limited to, cancer and an immune disease.

In the present disclosure, the cancer includes, but is not limited to, one or more of myeloproliferative neoplasm, lymphoma, and leukemia.

In the present disclosure, the immune disease includes, but is not limited to, one or more of rheumatoid arthritis, alopecia areata, atopic dermatitis, vitiligo, and psoriasis.

The present disclosure also provides a use of the crystal form II or crystal form III of the compound represented by formula 1 in the manufacture of a medicament for preventing and/or treating cancer or an immune disease.

In the present disclosure, the medicament may be used in combination with other therapeutic agents for preventing and/or treating cancer or an immune disease.

In the present disclosure, the other therapeutic agents may be used for preventing and/or treating cancer or an immune disease.

In the present disclosure, the cancer includes, but is not limited to, one or more of myeloproliferative neoplasm, lymphoma, and leukemia.

In the present disclosure, the immune disease includes, but is not limited to, one or more of rheumatoid arthritis, alopecia areata, atopic dermatitis, vitiligo, and psoriasis.

The present disclosure also provides a pharmaceutical composition comprising a therapeutically effective amount of the crystal form II and/or crystal form III of the compound represented by formula 1, and a pharmaceutically acceptable excipient.

In the present disclosure, the pharmaceutical composition may be formulated into various types of unit dosage forms, such as a tablet, a pill, a powder, a solution, an emulsion, an ointment, a capsule, or a liniment.

The present disclosure also provides a use of the pharmaceutical composition in the manufacture of a JAK kinase inhibitor.

The present disclosure also provides a use of the pharmaceutical composition in the manufacture of a medicament for preventing and/or treating a disease related to JAK kinase.

In the present disclosure, the medicament may be used in combination with other therapeutic agents for preventing and/or treating a disease related to JAK kinase.

In the present disclosure, the other therapeutic agents may be used for preventing and/or treating a disease related to JAK kinase.

In the present disclosure, the disease related to JAK kinase includes, but is not limited to, cancer and an immune disease.

In the present disclosure, the cancer includes, but is not limited to, one or more of myeloproliferative neoplasm, lymphoma, and leukemia.

In the present disclosure, the immune disease includes, but is not limited to, one or more of rheumatoid arthritis, alopecia areata, atopic dermatitis, vitiligo, and psoriasis.

The present disclosure also provides a use of the pharmaceutical composition in the manufacture of a medicament for preventing and/or treating cancer or an immune disease.

In the present disclosure, the medicament may be used in combination with other therapeutic agents for preventing and/or treating cancer or an immune disease.

In the present disclosure, the other therapeutic agents may be used for preventing and/or treating cancer or an immune disease.

In the present disclosure, the cancer includes, but is not limited to, one or more of myeloproliferative neoplasm, lymphoma, and leukemia.

In the present disclosure, the immune disease includes, but is not limited to, one or more of rheumatoid arthritis, alopecia areata, atopic dermatitis, vitiligo, and psoriasis.

The term “therapeutically effective amount” refers to an amount sufficient to effectively treat a disease when administered to a patient. The therapeutically effective amount will vary depending on the type of compound, the type of disease, the severity of the disease, the age of the patient, etc., but can be adjusted by those skilled in the art as appropriate.

The term “pharmaceutically acceptable excipient” refers to all substances contained in a pharmaceutical formulation other than the active pharmaceutical ingredient, and is generally divided into two categories: excipients and additives. For details, please refer to the Pharmacopoeia of the People's Republic of China (2020 Edition) and Handbook of Pharmaceutical Excipients (Paul J Sheskey, Bruno C Hancock, Gary P Moss, David J Goldfarb, 2020, 9th Edition).

In the present disclosure, “prevention/preventing” refers to a reduction in the risk of acquiring or developing a disease or disorder (i.e., causing at least one of the clinical symptoms of the disease not to develop in a subject who may be exposed to a disease-causing agent, or predisposed to the disease prior to the onset of the disease).

In the present disclosure, “treatment/treating” refers to ameliorating a disease or disorder (i.e., preventing the disease or reducing the extent or severity of its clinical symptoms); or ameliorating at least one physical parameter, which may not be discernible by the subject; or slowing the progression of the disease.

The crystal forms of the present disclosure can be identified by one or more solid analysis methods. For example, X-ray powder diffraction, single-crystal X-ray diffraction, infrared absorption spectroscopy, differential scanning calorimetry, thermogravimetric analysis. Those skilled in the art know that the peak intensity and/or peak situation of X-ray powder diffraction may vary due to different experimental conditions. At the same time, the measured 2θ value will have an error of about ±0.2° due to different accuracy of the instrument. The relative intensity value of the peak is more dependent on some properties of the sample being measured, such as the size and purity of the crystal, compared to the peak position. Therefore, the measured peak intensity may show a deviation of about ±20%. Despite experimental errors, instrumental errors, and preferential orientation, those skilled in the art can also obtain sufficient information for identifying crystal forms from the X-ray powder diffraction data provided in the present patent. In infrared spectroscopy, the shape of the spectrum and the position of the absorption peak will be affected to a certain extent due to differences in the performance of various types of instruments, and differences in the degree of grinding or the degree of moisture absorption in the preparation of the test sample. In DSC measurement, the initial temperature, peak temperature, and melting enthalpy data of the endothermic peak obtained by actual measurement are variable to a certain extent depending on the heating rate, the shape and purity of the crystal, and other measurement parameters.

In the present disclosure, “room temperature” refers to “10 to 30° C.”.

On the basis of not violating common sense in the art, the above preferred conditions can be combined arbitrarily to obtain preferred examples of the present disclosure.

The reagents and starting materials used in the present disclosure are all commercially available.

The positive and progressive effect of the present disclosure is that the crystal form II and crystal form III of the compound represented by formula 1 provided by the present disclosure have good physicochemical stability, are not prone to moisture absorption, and are of great value for the optimization and development of drugs.

### BRIEF DESCRIPTION OF THE DRAWINGS

FIG. 1 shows an X-ray powder diffraction pattern for crystal form I of the compound represented by formula 1.

FIG. 2 shows a differential scanning calorimetry pattern for crystal form I of the compound represented by formula 1.

FIG. 3 shows a thermogravimetric analysis pattern for crystal form I of the compound represented by formula 1.

FIG. 4 shows a dynamic vapor sorption pattern for crystal form I of the compound represented by formula 1.

FIG. 5 shows an X-ray powder diffraction pattern for crystal form II of the compound represented by formula 1.

FIG. 6 shows a differential scanning calorimetry pattern for crystal form II of the compound represented by formula 1.

FIG. 7 shows a thermogravimetric analysis pattern for crystal form II of the compound represented by formula 1.

FIG. 8 shows a dynamic vapor sorption pattern for crystal form II of the compound represented by formula 1.

FIG. 9 shows an infrared absorption spectrum for crystal form II of the compound represented by formula 1.

FIG. 10 shows an X-ray powder diffraction pattern for crystal form III of the compound represented by formula 1.

FIG. 11 shows a differential scanning calorimetry pattern for crystal form III of the compound represented by formula 1.

FIG. 12 shows a thermogravimetric analysis pattern for crystal form III of the compound represented by formula 1.

FIG. 13 shows a dynamic vapor sorption pattern for crystal form III of the compound represented by formula 1.

FIG. 14 shows an X-ray powder diffraction pattern for crystal form IV of the compound represented by formula 1.

FIG. 15 shows a differential scanning calorimetry pattern for crystal form IV of the compound represented by formula 1.

FIG. 16 shows a thermogravimetric analysis pattern for crystal form IV of the compound represented by formula 1.

FIG. 17 shows an X-ray powder diffraction pattern for crystal form V of the compound represented by formula 1.

FIG. 18 shows a differential scanning calorimetry pattern for crystal form V of the compound represented by formula 1.

FIG. 19 shows a thermogravimetric analysis pattern for crystal form V of the compound represented by formula 1.

FIG. 20 shows an X-ray powder diffraction pattern for crystal form VI of the compound represented by formula 1.

FIG. 21 shows a differential scanning calorimetry pattern for crystal form VI of the compound represented by formula 1.

### DETAILED DESCRIPTION OF THE PREFERRED EMBODIMENT

The present disclosure is further illustrated below by means of examples, but the present disclosure is not limited to the scope of the examples. The experimental methods for which specific conditions are not indicated in the following examples are selected according to conventional methods and conditions, or according to the product instructions.

### Example 1: Preparation of Compound Represented by Formula 1

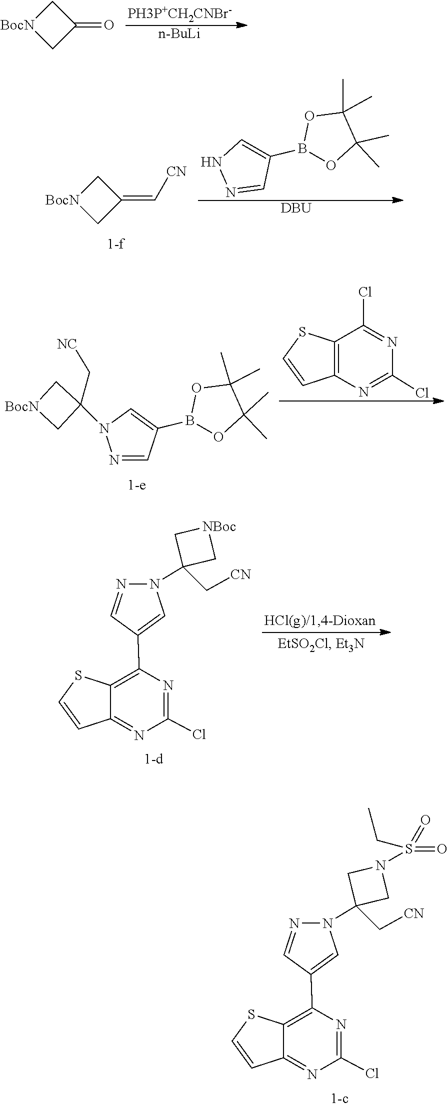

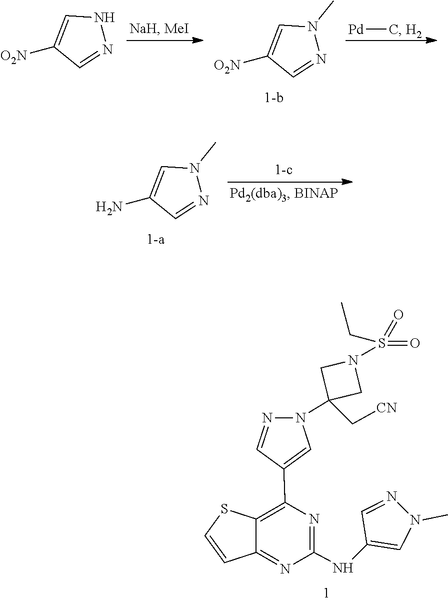

### Synthesis of Compound 1-f

A suspension of (cyanomethyl)triphenylphosphonium bromide (13.4 g, 35.09 mmol) in anhydrous tetrahydrofuran (100 mL) was cooled to 0° C. under a nitrogen atmosphere, and slowly added dropwise to a 2.5 M solution of n-butyllithium in n-hexane (15.5 mL, 38.59 mmol). The mixture was stirred at 0° C. for 30 minutes, then added with 1-Boc-3-azetidinone (6.0 g, 35.09 mmol), and warmed to room temperature and stirred for 1 hour. The reaction mixture was quenched with saturated ammonium chloride solution (50 mL) and extracted with ethyl acetate (150 mL×3). The organic phases were combined, sequentially washed with water (100 mL×3) and saturated brine (100 mL), dried over anhydrous sodium sulfate, filtered, and the filtrate was concentrated under reduced pressure. The residue was purified by silica gel column chromatography (petroleum ether/ethyl acetate=5:1) to obtain compound 1-f (2.5 g, yield: 37%) as a white solid. LC-MS (ESI): m/z=217 [M+Na]⁺.

### Synthesis of Compound 1-e

Compound 1-f (6.0 g, 30.93 mmol) and 4-pyrazoleboronic acid pinacol ester (9.2 g, 47.42 mmol) were dissolved in acetonitrile (60 mL), followed by the addition of 1,8-diazabicyclo[5.4.0]undec-7-ene (10.0 g, 65.79 mmol). The mixture was stirred at 60° C. for 18 hours. The reaction mixture was concentrated under reduced pressure. The residue was added with 1 N hydrochloric acid aqueous solution (100 mL) and extracted with ethyl acetate (100 mL×3). The organic phases were combined, sequentially washed with water (60 mL×3) and saturated brine (60 mL), dried over anhydrous sodium sulfate, filtered, and the filtrate was concentrated under reduced pressure. The residue was purified by silica gel column chromatography (petroleum ether/ethyl acetate=3:1) to obtain compound 1-e (7.1 g, yield: 59.2%) as a white solid. LC-MS (ESI): m/z=389 [M+H]⁺.

### Synthesis of Compound 1-d

Compound 1-e (4.0 g, 10.3 mmol), 2,4-dichlorothieno[3,2-d]pyrimidine (2.52 g, 12.4 mmol), and sodium carbonate (3.3 g, 31.2 mmol) were suspended in a mixed solvent of dioxane (25 mL) and water (25 mL) under a nitrogen atmosphere, then [1,1′-bis(diphenylphosphino)ferrocene]dichloropalladium(II) (1.1 g, 1.5 mmol) was added thereto, and the mixture was stirred at 80° C. for 16 hours. The reaction mixture was concentrated under reduced pressure. The residue was added with water (200 mL) and extracted with dichloromethane (200 mL×3). The organic phases were combined, sequentially washed with water (100 mL×3) and saturated brine (100 mL), dried over anhydrous sodium sulfate, filtered, and the filtrate was concentrated under reduced pressure. The residue was purified by silica gel column chromatography (petroleum ether/ethyl acetate=2:1) to obtain compound 1-d (3.2 g, yield: 63%) as a light yellow solid. LC-MS (ESI): m/z=431 [M+H]⁺.

### Synthesis of Compound 1-c

Compound 1-d (310 mg, 0.72 mmol) was dissolved in dichloromethane (2 mL), then a solution of hydrochloric acid in dioxane (4 N, 1 mL) was added thereto, and the mixture was stirred at room temperature for 16 hours. The reaction mixture was concentrated under reduced pressure. The residue was added with dichloromethane (10 mL) and triethylamine (2 mL). The mixture was cooled to 0° C., and ethylsulfonyl chloride (154 mg, 1.37 mmol) was slowly added dropwise thereto. After the dropwise addition was completed, the mixture was stirred at 0° C. for another 30 minutes, added with water (5 mL), and extracted with dichloromethane (10 mL×3). The organic phases were combined, sequentially washed with water (10 mL×3) and saturated brine (10 mL), dried over anhydrous sodium sulfate, filtered, and the filtrate was concentrated under reduced pressure. The residue was purified by silica gel column chromatography (petroleum ether/ethyl acetate=2:1) to obtain compound 1-c (108 mg, yield: 34%). LC-MS (ESI): m/z=423 [M+H]⁺.

### Synthesis of Compound 1-b

Sodium hydride (1.3 g, 32.1 mmol) was added to a solution of 4-nitropyrazole (3.3 g, 29.2 mmol) in anhydrous tetrahydrofuran (30 mL) at 0° C., and the mixture was stirred for 1 hour. Iodomethane (2 mL) was then slowly added thereto, and the mixture was stirred at room temperature for another 2 hours. The mixture was poured into ice water (100 mL) and extracted with ethyl acetate (50 mL×3). The organic phase was dried over anhydrous sodium sulfate, filtered, and the filtrate was concentrated under reduced pressure. The residue was added with a mixed solvent of petroleum ether and ethyl acetate (20:1, 20 mL), and stirred to precipitate a solid. After filtration, the solid was dried under vacuum for 8 hours to obtain compound 1-b (2.6 g, yield: 70%) as a white solid. The product was directly used in the next reaction step without further purification. LC-MS (ESI): m/z=128 [M+H]⁺.

### Synthesis of Compound 1-a

10% palladium on carbon (0.2 g) was added to a solution of compound 15-b (1.0 g, 7.87 mmol) in ethanol (15 mL) under a hydrogen atmosphere. The mixture was reacted at 25° C. for 18 hours, filtered, and the filtrate was concentrated under reduced pressure. The residue was purified by silica gel column chromatography (petroleum ether/ethyl acetate=1:1) to obtain compound 1-a (700 mg, yield: 92%) as a red oil.

### Synthesis of Compound 1

Tris(dibenzylideneacetone)dipalladium (55 mg, 0.06 mmol) and 2,2′-bis(diphenylphosphino)-1,1′-binaphthyl (40 mg, 0.06 mmol) were added to a suspension of compound 1-c, compound 1-a (138 mg, 1.42 mmol), and cesium carbonate (309 mg, 0.95 mmol) in dioxane (4 mL) under a nitrogen atmosphere, and the mixture was reacted under microwave irradiation at 120° C. for 60 minutes. The reaction mixture was cooled to room temperature, diluted with dichloromethane (20 mL), filtered, and the filtrate was concentrated under reduced pressure. The residue was subjected to preparative high-performance liquid chromatography (mobile phase: acetonitrile, water (0.05% trifluoroacetic acid); gradient: 60%-90%-10%) to obtain crystal form I of compound 1 (23 mg, yield: 14%) as a light yellow solid. LC-MS (ESI): m/z=484 [M+H]⁺.

### Example 2: Preparation of Crystal Form II of Compound Represented by Formula 1

### Method 1:

20 mg of crystal form I of the compound represented by formula 1, obtained according to the preparation method in Example 1, was weighed into a glass vial. 20 times the volume of acetonitrile as solvent was added to the vial, and the mixture was sonicated for 1 minute to obtain a suspension. The vial containing the suspension was wrapped in aluminum foil to be protected from light and placed on a Labquaker rotator. The mixture was rotated 360 degrees at room temperature (approximately 25° C.) for equilibration. During the equilibration process, a sample (0.8 mL) was taken and centrifuged, and the remaining solid was naturally dried. The resulting sample was characterized by XRPD as crystal form II.

### Method 2:

20 mg of the compound represented by formula 1 was weighed into a glass vial, which was placed in a 50° C. water bath with a rotational speed of 400 rpm. 30 times the volume of acetone was added to the vial, and the mixture was heated to obtain a clarified solution. The sample solution was filtered through a 0.45 μm filter membrane while still hot, and the filtrate was transferred to a 5 mL centrifuge tube, which was immediately placed in a −20° C. freezer and stored overnight. After centrifugation, the solid was collected, naturally dried, and characterized by XRPD as crystal form II.

### Method 3:

20 mg of the compound represented by formula 1 was weighed into a glass vial, which was placed in a 50° C. water bath with a rotational speed of 400 rpm. 90 times the volume of ethyl acetate as good solvent was added to the vial, and the mixture was heated to obtain a clarified solution, which was maintained at the same temperature for 15 minutes. 70 times the volume of n-heptane as anti-solvent was slowly added dropwise thereto under stirring to precipitate a solid, and the mixture was stirred for another 10 minutes. A sample was taken and centrifuged, naturally dried, and characterized by XRPD as crystal form II. After 8 days, the sample was characterized by XRPD and remained as crystal form II.

Following the same operational procedure as in method 3, crystal form II was also obtained by replacing ethyl acetate with the good solvent listed in the table below.

| | Times | Times the | | XRPD |
|---|---|---|---|---|
| | the | volume of | | characterization |
| Good solvent | volume | n-heptane | Time | results |
| Acetone | 30 | 30 | 10 minutes | Crystal form II |
| | | | (1) | |
| | | | 8 days | Crystal form II |
| | | | (2) | |
| Dichloromethane | 30 | 50 | 10 minutes | Crystal form II |
| | | | (1) | |
| | | | 8 days | Crystal form II |
| | | | (2) | |
| Tetrahydrofuran | 30 | 50 | 10 minutes | Crystal form II |
| | | | (1) | |
| | | | 8 days | Crystal form II |
| | | | (2) | |

Following the same operational procedure as in method 3, crystal form II was also obtained by using the good solvent and anti-solvent listed in the table below. After 6 days, the sample was characterized by XRPD and remained as crystal form II.

| | | Times the | | |
|---|---|---|---|---|
| | Times | volume of | | |
| | the | methyl tert- | | Characteriza- |
| Good solvent | volume | butyl ether | Time | tion results |
| Ethyl acetate | 180 | 530 | 10 minutes | Crystal form II |
| | | | (1) | |
| | | | 6 days | Crystal form II |
| | | | (2) | |
| Acetone | 50 | 210 | 10 minutes | Crystal form II |
| | | | (1) | |
| | | | 6 days | Crystal form II |
| | | | (2) | |
| Dichloromethane | 50 | 50 | 10 minutes | Crystal form II |
| | | | (1) | |
| | | | 6 days | Crystal form II |
| | | | (2) | |
| Tetrahydrofuran | 50 | 160 | 10 minutes | Crystal form II |
| | | | (1) | |
| | | | 6 days | Crystal form II |
| | | | (2) | |

### Example 3: Preparation of Crystal Form III of Compound Represented by Formula 1

50 mg of the compound represented by formula 1, obtained according to the preparation method in Example 1, was weighed into a glass vial. 100 times the volume of a mixed solvent of methanol/water (volume ratio=10:1) was added to the vial, and the mixture was sonicated for 1 minute to obtain a suspension, which was placed in a 50° C. water bath and stirred for 4 hours. 25 times the volume of water was slowly added dropwise thereto, and the mixture was maintained at the same temperature and stirred overnight. The next day, the mixture was cooled to room temperature, stirred for another 3 days, and dried under vacuum at 45° C. The resulting sample was characterized by XRPD as crystal form III.

### Example 4: Preparation of Crystal Form IV of Compound Represented by Formula 1

20 mg of the compound represented by formula 1, obtained according to the preparation method in Example 1, was weighed into a glass vial. An appropriate volume of dichloromethane was added to the vial, and the mixture was sonicated to dissolve the compound, resulting in a saturated solution of the compound represented by formula 1. After complete dissolution, the mixture was sonicated for another 5 minutes. The clarified solution was filtered through a 0.45 μm filter membrane, and the filtrate was transferred to a 5 mL centrifuge tube. The glass vial was wrapped in aluminum foil to be protected from light and exposed to open conditions at room temperature (approximately 25° C.) to allow the solvent to evaporate naturally. The resulting sample was characterized by XRPD as crystal form IV (a dichloromethane solvate).

### Example 5: Preparation of Crystal Form V of Compound Represented by Formula 1

20 mg of the compound represented by formula 1, obtained according to the preparation method in Example 1, was weighed into a glass vial. An appropriate volume of tetrahydrofuran was added to the vial, and the mixture was sonicated to dissolve the compound, resulting in a saturated solution of the compound represented by formula 1. After complete dissolution, the mixture was sonicated for another 5 minutes. The clarified solution was filtered through a 0.45 μm filter membrane, and the filtrate was transferred to a 10 mL glass vial. The glass vial was wrapped in aluminum foil to be protected from light and exposed to open conditions at room temperature (approximately 25° C.) to allow the solvent to evaporate naturally. The resulting sample was characterized by XRPD as crystal form V (a tetrahydrofuran solvate).

### Example 6: Preparation of Crystal Form VI of Compound Represented by Formula 1

20 mg of the compound represented by formula 1, obtained according to the preparation method in Example 1, was weighed into a glass vial. An appropriate volume of 1,4-dioxane was added to the vial, and the mixture was sonicated to dissolve the compound, resulting in a saturated solution of the compound represented by formula 1. After complete dissolution, the mixture was sonicated for another 5 minutes. The clarified solution was filtered through a 0.45 μm filter membrane, and the filtrate was transferred to a 5 mL centrifuge tube. The glass vial was wrapped in aluminum foil to be protected from light and exposed to open conditions at room temperature (approximately 25° C.) to allow the solvent to evaporate naturally. The resulting sample was characterized by XRPD as crystal form VI (a 1,4-dioxane solvate).

### Example 7: X-Ray Powder Diffraction (XRPD)

Method: An appropriate amount of sample was taken and spread evenly on a single-crystal silicon wafer, and XRPD testing was conducted at room temperature. The specific experimental parameters were as follows: the light source was CuKα, the X-ray intensity was 40 kV/40 mA, the scanning mode was Theta-theta, the scanning angle range was 4° to 40°, the step size was 0.05°, and the scanning speed was 0.5 seconds per step.

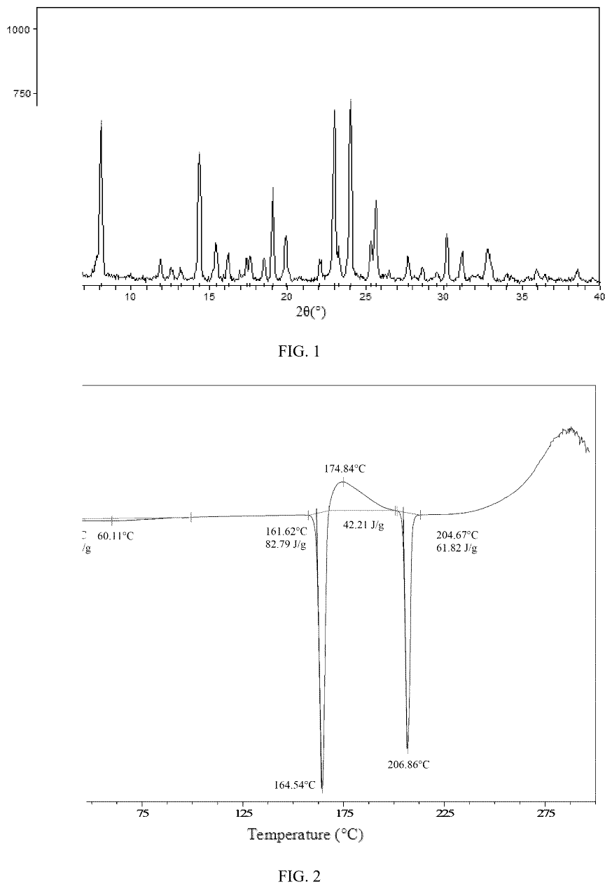

The X-ray powder diffraction pattern for crystal form I of the compound represented by formula 1, prepared according to Example 1, is shown in FIG. 1, in which the 2θ angles of the characteristic diffraction peaks are 6.599°±0.2°, 8.086°±0.2°, 11.879°±0.2°, 12.589°±0.2°, 13.184°±0.2°, 14.375°±0.2°, 15.434°±0.2°, 16.213°±0.2°, 17.372°±0.2°, 17.618°±0.2°, 18.502°±0.2°, 19.066°±0.2°, 19.897°±0.2°, 22.074°±0.2°, 22.997°±0.2°, 23.240°±0.2°, 24.033°±0.2°, 25.339°±0.2°, 25.641°±0.2°, 27.694°±0.2°, 28.640°±0.2°, 29.540°±0.2°, 30.179°±0.2°, 31.164°±0.2°, 32.816°±0.2°, 34.022°±0.2°, 35.920°±0.2°, 36.472°±0.2°, and 38.536°±0.2°.

The X-ray powder diffraction pattern for crystal form II of the compound represented by formula 1, prepared according to Example 2, is shown in FIG. 5, in which the 2θ angles of the characteristic diffraction peaks are 6.976°±0.2°, 8.928°±0.2°, 10.781°±0.2°, 12.775°±0.2°, 13.316°±0.2°, 13.967°±0.2°, 16.220°±0.2°, 16.450°±0.2°, 16.817°±0.2°, 17.598°±0.2°, 19.377°±0.2°, 19.494°±0.2°, 19.955°±0.2°, 20.875°±0.2°, 22.119°±0.2°, 24.798°±0.2°, 25.026°±0.2°, 25.752°±0.2°, 26.778°±0.2°, 27.959°±0.2°, 28.541°±0.2°, 28.957°±0.2°, 30.160°±0.2°, 33.201°±0.2°, 34.555°±0.2°, 34.916°±0.2°, 36.340°±0.2°, 36.602°±0.2°, and 37.986°±0.2°.

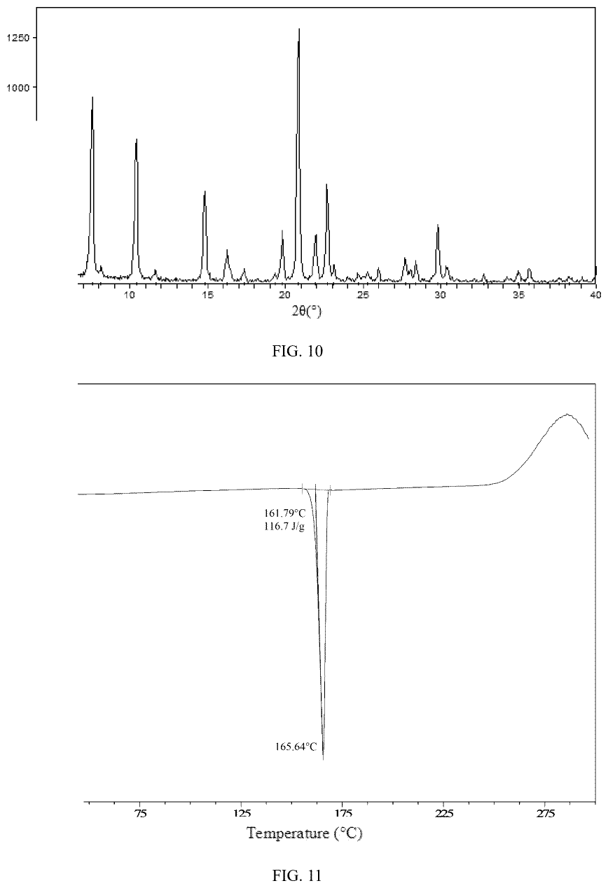

The X-ray powder diffraction pattern for crystal form III of the compound represented by formula 1, prepared according to Example 3, is shown in FIG. 10, in which the 2θ angles of the characteristic diffraction peaks are 5.515°±0.2°, 7.577°±0.2°, 8.137°±0.2°, 10.415°±0.2°, 11.632°±0.2°, 14.809°±0.2°, 16.258°±0.2°, 17.347°±0.2°, 19.318°±0.2°, 19.797°±0.2°, 20.813°±0.2°, 21.939°±0.2°, 22.663°±0.2°, 23.108°±0.2°, 24.664°±0.2°, 25.282°±0.2°, 25.998°±0.2°, 27.689°±0.2°, 28.387°±0.2°, 29.791°±0.2°, 30.347°±0.2°, 32.742°±0.2°, 34.932°±0.2°, 35.679°±0.2°, 37.573°±0.2°, and 38.218°±0.2°.

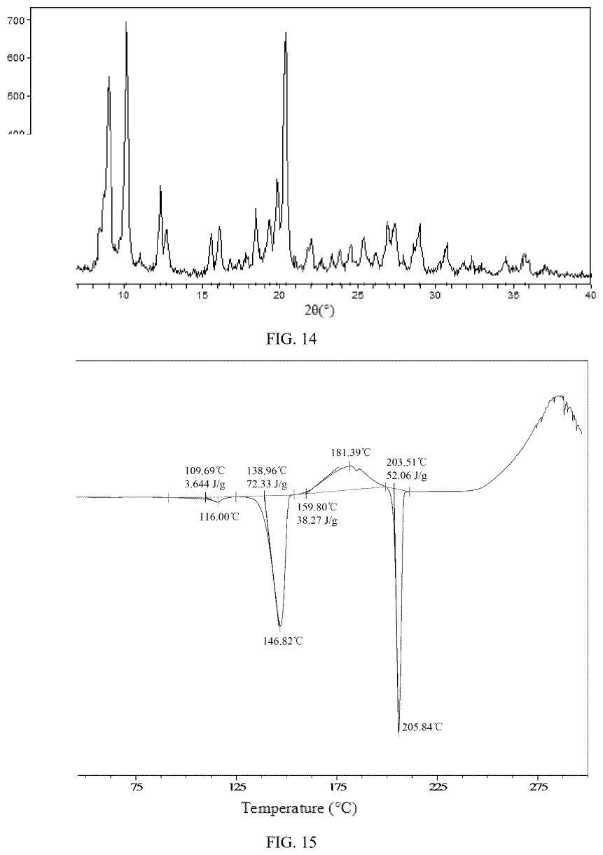

The X-ray powder diffraction pattern for crystal form IV of the compound represented by formula 1, prepared according to Example 4, is shown in FIG. 14, in which the 2θ angles of the characteristic diffraction peaks are 8.994°±0.2°, 10.136°±0.2°, 12.324°±0.2°, 12.684°±0.2°, 15.595°±0.2°, 16.110°±0.2°, 16.816°±0.2°, 18.461°±0.2°, 19.309°±0.2°, 19.815°±0.2°, 20.351°±0.2°, 22.032°±0.2°, 23.327°±0.2°, 23.846°±0.2°, 24.526°±0.2°, 25.386°±0.2°, 26.121°±0.2°, 26.896°±0.2°, 27.381°±0.2°, 28.970°±0.2°, and 30.712°±0.2°.

The X-ray powder diffraction pattern for crystal form V of the compound represented by formula 1, prepared according to Example 5, is shown in FIG. 17, in which the 2θ angles of the characteristic diffraction peaks are 5.486°±0.2°, 7.138°±0.2°, 8.075°±0.2°, 9.609°±0.2°, 10.355°±0.2°, 10.678°±0.2°, 10.965°±0.2°, 11.570°±0.2°, 14.764°±0.2°, 15.685°±0.2°, 16.221°±0.2°, 16.836°±0.2°, 17.169°±0.2°, 17.270°±0.2°, 17.350°±0.2°, 17.881°±0.2°, 18.117°±0.2°, 19.215°±0.2°, 19.712°±0.2°, 20.796°±0.2°, 21.253°±0.2°, 21.590°±0.2°, 21.902°±0.2°, 22.726°±0.2°, 23.095°±0.2°, 24.946°±0.2°, 25.151°±0.2°, 25.344°±0.2°, 25.985°±0.2°, 28.011°±0.2°, 28.294°±0.2°, and 29.713°±0.2°.

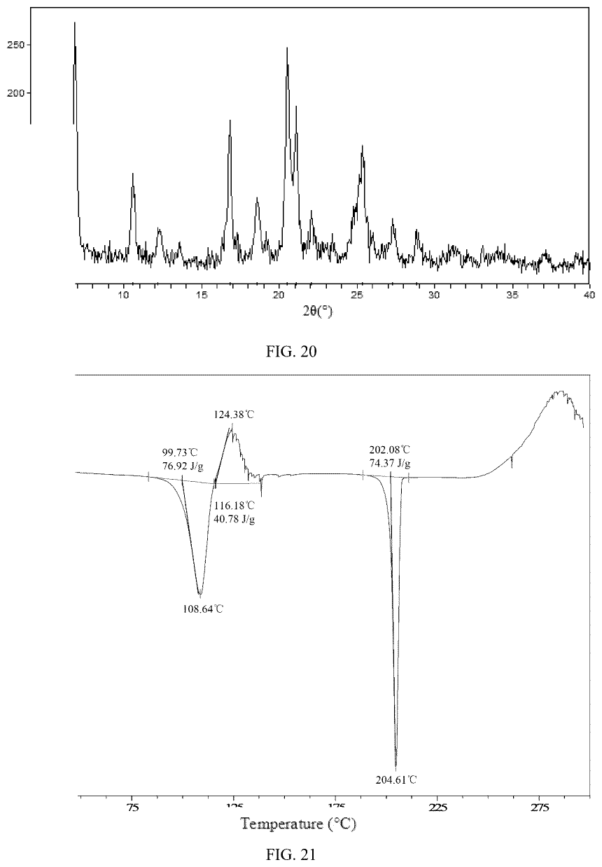

The X-ray powder diffraction pattern for crystal form VI of the compound represented by formula 1, prepared according to Example 6, is shown in FIG. 20, in which the 2θ angles of the characteristic diffraction peaks are 6.808°±0.2°, 10.582°±0.2°, 12.200°±0.2°, 16.811°±0.2°, 18.568°±0.2°, 20.510°±0.2°, 21.094°±0.2°, 22.062°±0.2°, 25.346°±0.2°, 27.293°±0.2θ, and 28.831°±0.2°.

### Example 8: Infrared Absorption Spectroscopy (IR)

According to the infrared spectrophotometric method in General Rule 0402 of the Pharmacopoeia of the People's Republic of China (2015 Edition, Volume IV), the sample was prepared using the KBr pellet method, and the infrared absorption spectra were collected in the wave number range of 4000 to 400 cm⁻¹. The number of scans for the sample was 45, and the resolution of the instrument was 4 cm⁻¹.

The infrared absorption spectrum for crystal form II of the compound represented by formula 1 is shown in FIG. 9, in which the characteristic peaks, vibration types, functional groups, and absorption peak intensities are also shown in Table 1 below.

TABLE 1

| | | | Absorption |
|---|---|---|---|
| Absorption peak wave | Assigned functional group and | Functional | peak |
| number (cm⁻¹) | vibration type | group | intensity |
| 3375 | N—H stretching vibration | —NHR | S |
| 3105, 2966, 2922 | C—H stretching vibration | C═C—H, | m |
| | | —CH₂, —CH₃ | |
| 1651, 1595, 1577, 1523, | C═C, C═N stretching vibration and | Aromatic ring | S |
| 1446 | aromatic ring skeleton vibration | | |
| 1479, 1382 | C—H bending vibration | —CH₂, —CH₃ | S |
| 1342, 1139, 1020 | C—N stretching vibration | —C—N— | S |
| 883, 686 | ═C—H bending vibration | ═C—H | m |

### Example 9: Differential Scanning Calorimetry (DSC)

A sample of crystal form I of the compound represented by formula 1 (2.8790 mg) was weighed into a non-sealed aluminum pan. The sample was equilibrated at 25° C. under a nitrogen flow (50 mL/min) atmosphere, and then heated from 25° C. to 300° C. at a heating rate of 10° C./min. The melting enthalpy was 82.79 J/g at a temperature of 161.62° C. to 164.54° C. and 61.82 J/g at a temperature of 204.67° C. to 206.86° C., as shown in FIG. 2.

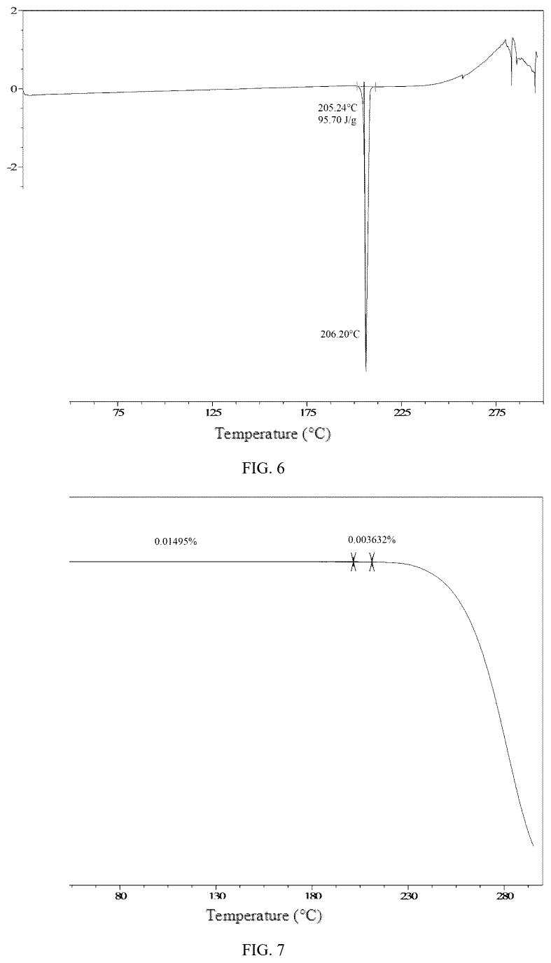

A sample of crystal form II of the compound represented by formula 1 (2.3250 mg) was weighed into a non-sealed aluminum pan. The sample was equilibrated at 25° C. under a nitrogen flow (50 mL/min) atmosphere, and then heated from 25° C. to 300° C. at a heating rate of 10° C./min. The melting enthalpy was 95.70 J/g at a temperature of 205.24° C. to 206.20° C., as shown in FIG. 6.

A sample of crystal form III of the compound represented by formula 1 (1.4970 mg) was weighed into a non-sealed aluminum pan. The sample was equilibrated at 25° C. under a nitrogen flow (50 mL/min) atmosphere, and then heated from 25° C. to 300° C. at a heating rate of 10° C./min. The melting enthalpy was 116.7 J/g at a temperature of 161.79° C. to 165.64° C., as shown in FIG. 11.

A sample of crystal form IV of the compound represented by formula 1 (2.0230 mg) was weighed into a non-sealed aluminum pan. The sample was equilibrated at 25° C. under a nitrogen flow (50 mL/min) atmosphere, and then heated from 25° C. to 300° C. at a heating rate of 10° C./min. A small solvent peak appeared before 125° C., which should correspond to the solvent dichloromethane. The melting enthalpy was 72.33 J/g at a temperature of 138.96° C. to 146.82° C. and 52.06 J/g at a temperature of 203.51° C. to 205.84° C., as shown in FIG. 15.

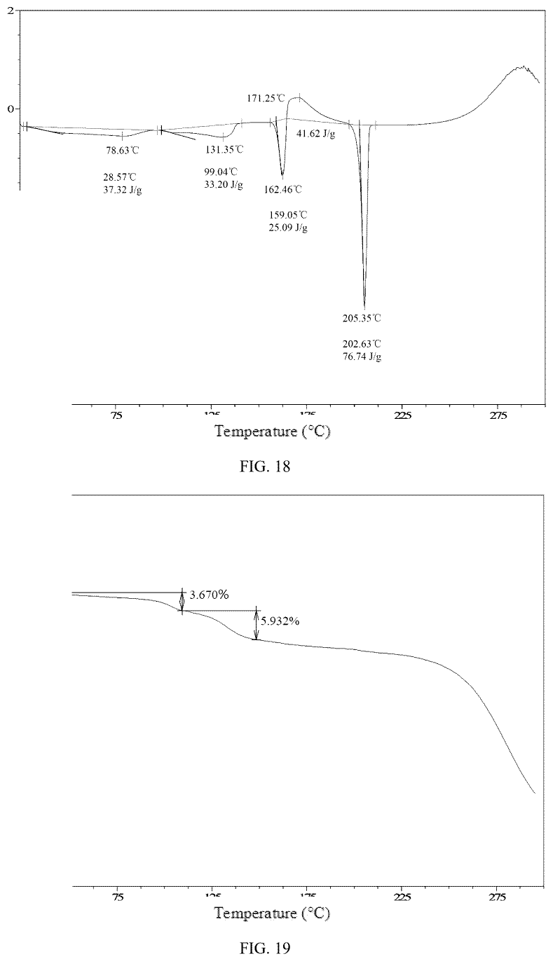

A sample of crystal form V of the compound represented by formula 1 (1.5680 mg) was weighed into a non-sealed aluminum pan. The sample was equilibrated at 25° C. under a nitrogen flow (50 mL/min) atmosphere, and then heated from 25° C. to 300° C. at a heating rate of 10° C./min. Two small absorption peaks appeared before 145° C. The melting enthalpy was 25.09 J/g at a temperature of 159.05° C. to 162.46° C. and 76.74 J/g at a temperature of 202.63° C. to 205.35° C., as shown in FIG. 18.

A sample of crystal form VI of the compound represented by formula 1 (1.5480 mg) was weighed into a non-sealed aluminum pan. The sample was equilibrated at 25° C. under a nitrogen flow (50 mL/min) atmosphere, and then heated from 25° C. to 300° C. at a heating rate of 10° C./min. Two small solvent absorption peaks appeared before 125° C. The melting enthalpy was 74.37 J/g at a temperature of 202.08° C. to 204.61.46° C., as shown in FIG. 21.

### Example 10: Thermogravimetric Analysis (TGA)

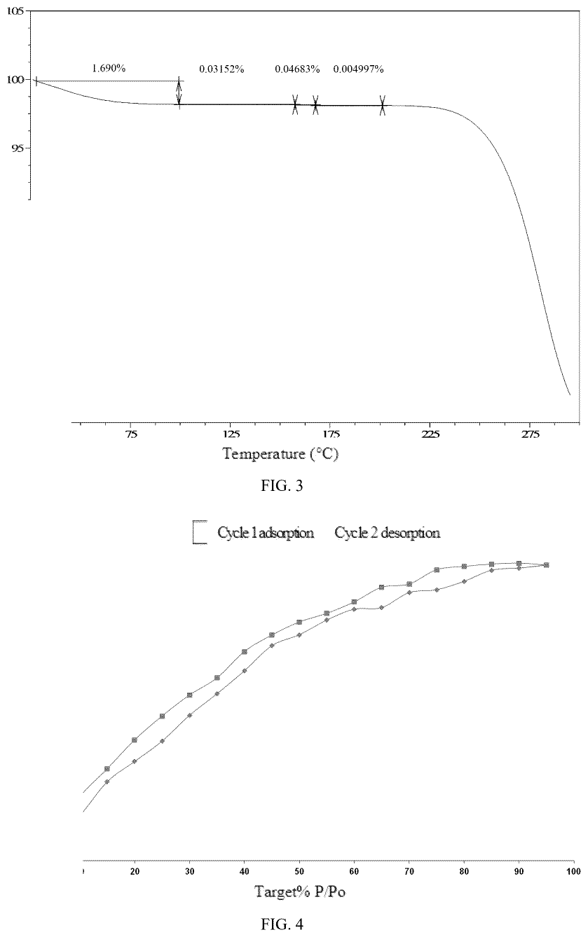

A sample of crystal form I of the compound represented by formula 1 (8.1060 mg) was weighed into a platinum sample pan. The sample was heated from 25° C. to 300° C. at a heating rate of 10° C./min under a nitrogen flow (50 mL/min) atmosphere, as shown in FIG. 3. A weight loss of 1.690% was observed from 25° C. to 100° C., which may be attributed to a small amount of solvent or water in the sample. Almost no weight loss was observed from 100° C. to the melting point.

A sample of crystal form II of the compound represented by formula 1 (10.4510 mg) was weighed into a platinum sample pan. The sample was heated from 25° C. to 300° C. at a heating rate of 10° C./min under a nitrogen flow (50 mL/min) atmosphere, as shown in FIG. 7. A weight loss of only 0.01495% was observed from 25° C. to the melting point, indicating that there was almost no residual solvent or water in the sample.

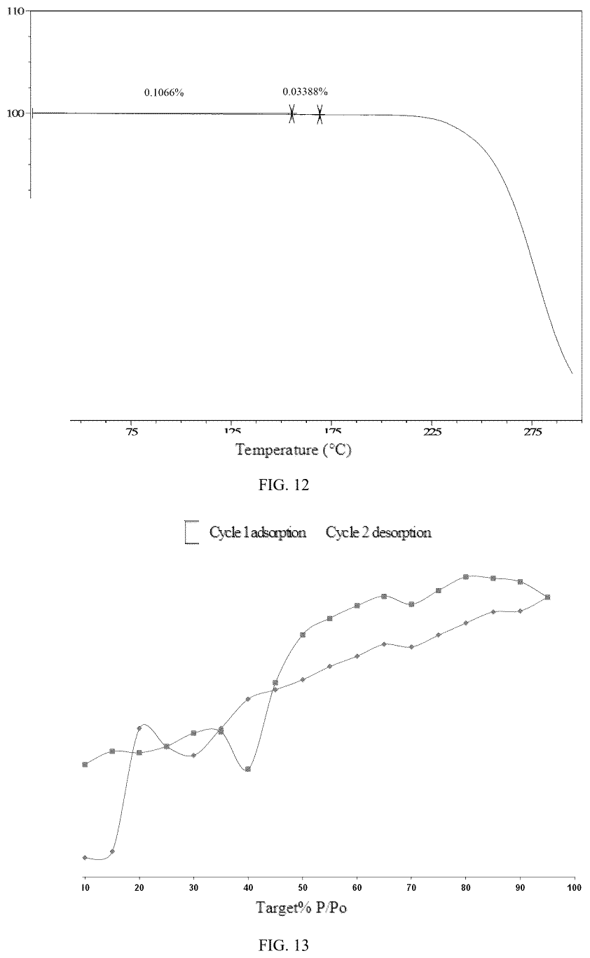

A sample of crystal form III of the compound represented by formula 1 (5.1880 mg) was weighed into a platinum sample pan. The sample was heated from 25° C. to 300° C. at a heating rate of 10° C./min under a nitrogen flow (50 mL/min) atmosphere, as shown in FIG. 12. A weight loss of only 0.1066% was observed from 25° C. to the melting point, indicating that there was almost no residual solvent or water in the sample.

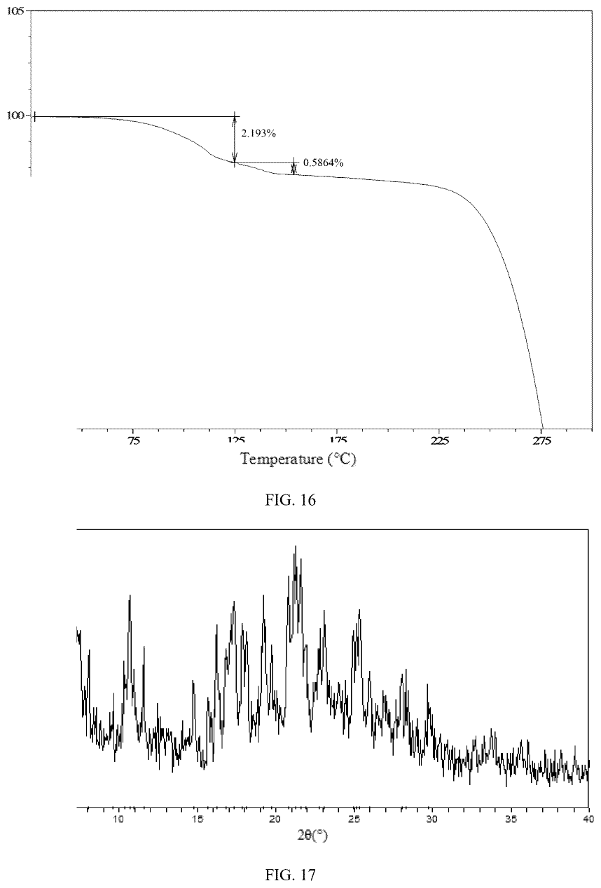

A sample of crystal form IV of the compound represented by formula 1 (4.6640 mg) was weighed into a platinum sample pan. The sample was heated from 25° C. to 300° C. at a heating rate of 10° C./min under a nitrogen flow (50 mL/min) atmosphere, as shown in FIG. 16. A weight loss of 2.193% was observed from 25° C. to 125° C., which should correspond to the solvent dichloromethane.

A sample of crystal form V of the compound represented by formula 1 (2.1270 mg) was weighed into a platinum sample pan. The sample was heated from 25° C. to 300° C. at a heating rate of 10° C./min under a nitrogen flow (50 mL/min) atmosphere, as shown in FIG. 19. A weight loss of 3.670% was observed from 25° C. to 95° C., which should correspond to free tetrahydrofuran. A weight loss of 5.932% was observed from 95° C. to 145° C., corresponding to the solvent peak in the DSC, which should be attributed to tetrahydrofuran incorporated into the crystal lattice.

### Example 11: Dynamic Vapor Sorption (DVS)

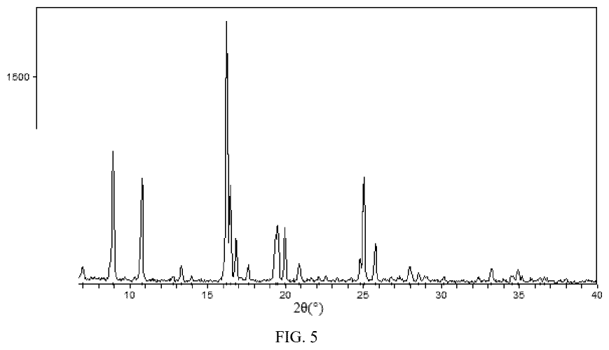

A sample of crystal form I of the compound represented by formula 1 (10 mg) was weighed, and dried for 60 minutes at a temperature of 25° C. and a humidity of 0% RH. The moisture absorption characteristics of the sample were tested as the humidity changed from 0% RH to 95% RH, and the desorption characteristics of the sample were tested as the humidity changed from 95% RH to 0% RH. The humidity change in each step was 5% RH, with an equilibrium criterion of a weight change rate of less than 0.01%/min within 5 minutes and a maximum equilibration time of 2 hours. The results showed a weight gain of 3.025% from 0% RH to 95% RH, as shown in FIG. 4.

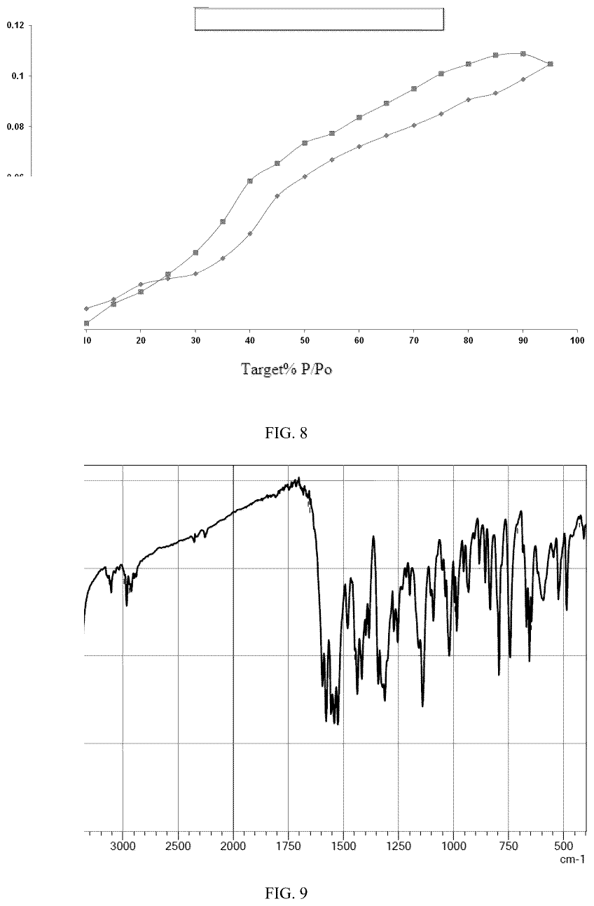

A sample of crystal form II of the compound represented by formula 1 (10 mg) was weighed, and dried for 60 minutes at a temperature of 25° C. and a humidity of 0% RH. The moisture absorption characteristics of the sample were tested as the humidity changed from 0% RH to 95% RH, and the desorption characteristics of the sample were tested as the humidity changed from 95% RH to 0% RH. The humidity change in each step was 5% RH, with an equilibrium criterion of a weight change rate of less than 0.01%/min within 5 minutes and a maximum equilibration time of 2 hours. The results showed a weight gain of 0.1047% from 0% RH to 95% RH, as shown in FIG. 8.

A sample of crystal form III of the compound represented by formula 1 (10 mg) was weighed, and dried for 60 minutes at a temperature of 25° C. and a humidity of 0% RH. The moisture absorption characteristics of the sample were tested as the humidity changed from 0% RH to 95% RH, and the desorption characteristics of the sample were tested as the humidity changed from 95% RH to 0% RH. The humidity change in each step was 5% RH, with an equilibrium criterion of a weight change rate of less than 0.01%/min within 5 minutes and a maximum equilibration time of 2 hours. The results showed a weight gain of 0.4084% from 0% RH to 95% RH, as shown in FIG. 13.

### Example 12: Stability Test

Appropriate amounts of crystal form II and crystal form III of the compound represented by formula 1 were accurately weighed into 20 mL colorless transparent glass vials, which were placed under corresponding influencing factors (high temperature at 60° C., high humidity of 92.5% RH) and accelerated conditions (40° C./75% RH) respectively. After 1 week and 2 weeks, the samples were removed for HPLC analysis to determine the content and related substances, thereby evaluating the chemical stability of crystal form II and crystal form III. The samples were also characterized by appearance, XRPD, and DSC to assess the physical stability of the two crystal forms. Additionally, samples of crystal form II and crystal form III were accurately weighed into 20 mL colorless transparent glass vials, which were tightly sealed and stored in a −20° C. freezer as standard samples for HPLC analysis. The test results are shown in Tables 2 and 3 below:

TABLE 2

| | | | Stability | |
|---|---|---|---|---|
| Group | Sample name | Stability test conditions | test time | [symbol] |
| Control group | Crystal form II | −20° C. | 2 weeks | [symbol] |
| Experimental group | Crystal form II | High temperature (60° C.) | 1 week | [symbol] |
| | | | 2 weeks | [symbol] |
| | | High humidity (92.5% RH) | 1 week | [symbol] |
| | | | 2 weeks | [symbol] |
| | | Accelerated (40° C./75%) | 1 week | [symbol] |
| | | | 2 weeks | [symbol] |
| Control group | Crystal form III | −20° C. | 2 weeks | [symbol] |
| Experimental group | Crystal form III | High temperature (60° C.) | 1 week | [symbol] |
| | | | 2 weeks | [symbol] |
| | | High humidity (92.5% RH) | 1 week | [symbol] |
| | | | 2 weeks | [symbol] |
| | | Accelerated (40° C./75%) | 1 week | [symbol] |
| | | | 2 weeks | [symbol] |

Note:
“/” in the table means no change. [symbol]
indicates data missing or illegible when filed

Note: “/” in the table means no change.

TABLE 3

| | | | Characterization | |
|---|---|---|---|---|
| | | | results | |
| Group | Sample name | Stability test conditions | Appearance | [symbol] |
| Control group | Crystal form II | −20° C. | Yellow | [symbol] |
| Experimental group | Crystal form II | High temperature (60° C.) | Yellow | [symbol] |
| | | High humidity (92.5% RH) | Yellow | [symbol] |
| | | Accelerated (40° C./75%) | Yellow | [symbol] |
| Control group | Crystal form III | −20° C. | Bright yellow | [symbol] |
| Experimental group | Crystal form III | High temperature (60° C.) | Bright yellow | [symbol] |
| | | High humidity (92.5% RH) | Bright yellow | [symbol] |
| | | Accelerated (40° C./75%) | Bright yellow | [symbol] |

[symbol]

indicates data missing or illegible when filed

The results of the stability test showed that crystal form III has good chemical stability after 2 weeks under −20° C., high temperature, high humidity, light, and accelerated conditions. However, partial conversion of crystal form III to crystal form II was observed under these conditions. Crystal form II has good physicochemical stability under −20° C., high temperature, high humidity, and accelerated conditions.

Additionally, crystal form II showed a weight gain of only 0.1047% from 0% RH to 95% RH, indicating almost no hygroscopicity.

What is claimed is:

1. Crystal form II or crystal form III of a compound represented by formula 1, wherein the crystal form II has an X-ray powder diffraction pattern using CuKα radiation and represented by 2θ angles comprising characteristic peaks at 8.928°±0.2°, 10.781°±0.2°, 16.220°±0.2°, 16.817°±0.2°, 19.494°±0.2°, 19.955°±0.2°, and 25.026°±0.2°; and the crystal form III has an X-ray powder diffraction pattern using CuKα radiation and represented by 2θ angles comprising characteristic peaks at 7.577°±0.2°, 10.415°±0.2°, 14.809°±0.2°, 19.797°±0.2°, 20.813°±0.2°, 21.939°±0.2°, 22.663°±0.2°, 27.689°±0.2°, and 29.791°±0.2°;

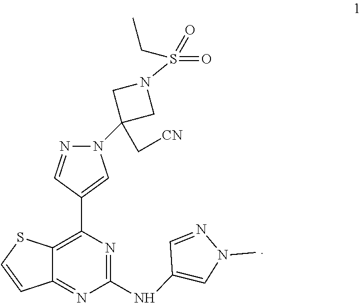 [SMILES: CCS(=O)(=O)N1CC(CC#N)(n2cc(-c3nc(Nc4cnn(C)c4)nc4ccsc34)cn2)C1]

2. The crystal form II or the crystal form III of the compound represented by formula 1 according to claim 1, wherein the crystal form II satisfies one or more of the following conditions: (1) the X-ray powder diffraction pattern for the crystal form II of the compound represented by formula 1, using CuKα radiation and represented by 2θ angles, further comprises characteristic peaks at one or more of 6.976°±0.2°, 12.775°±0.2°, 13.316°±0.2°, 13.967°±0.2°, 16.450°±0.2°, 17.598°±0.2°, 19.377°±0.2°, 20.875°±0.2°, 22.119°±0.2°, 24.798°±0.2°, 25.752°±0.2°, 26.778°±0.2°, 27.959°±0.2°, 28.541°±0.2°, 28.957°±0.2°, 30.160°±0.2°, 33.201°±0.2°, 34.555°±0.2°, 34.916°±0.2°, 36.340°±0.2°, 36.602°±0.2°, and 37.986°±0.2°;
(2) the crystal form II of the compound represented by formula 1 has a differential scanning calorimetry pattern comprising an absorption peak at 205° C.±5° C., with a melting enthalpy of 95.70 J/g;
(3) the crystal form II of the compound represented by formula 1 has a thermogravimetric analysis pattern with a weight loss of 0.01495% from 25° C. to the melting point, where the “%” represents weight percentage;
(4) the crystal form II of the compound represented by formula 1 has a dynamic vapor sorption pattern with a moisture absorption weight gain of 0.1047% in the range of 0% to 95% relative humidity, where the “%” represents weight percentage;
(5) the crystal form II of the compound represented by formula 1 has an infrared absorption spectrum determined by KBr pellet method comprising characteristic peaks at 3375 cm⁻¹, 3105 cm⁻¹, 2966 cm⁻¹, 2922 cm⁻¹, 1651 cm⁻¹, 1595 cm⁻¹, 1577 cm⁻¹, 1523 cm⁻¹, 1446 cm⁻¹, 1479 cm⁻¹, 1382 cm⁻¹, 1342 cm⁻¹, 1139 cm⁻¹, 1020 cm⁻¹, 883 cm⁻¹, and 686 cm⁻¹; and
the crystal form III satisfies one or more of the following conditions:
(1) the X-ray powder diffraction pattern for the crystal form III of the compound represented by formula 1, using CuKα radiation and represented by 2θ angles, further comprises characteristic peaks at one or more of 5.515°±0.2°, 8.137°±0.2°, 11.632°±0.2°, 16.258°±0.2°, 17.347°±0.2°, 19.318°±0.2°, 23.108°±0.2°, 24.664°±0.2°, 25.282°±0.2°, 25.998°±0.2°, 28.387°±0.2°, 30.347°±0.2°, 32.742°±0.2°, 34.932°±0.2°, 35.679°±0.2°, 37.573°±0.2°, and 38.218°±0.2°;
(2) the crystal form III of the compound represented by formula 1 has a differential scanning calorimetry pattern comprising an absorption peak at 162° C.±5° C., with a melting enthalpy of 116.7 J/g;
(3) the crystal form III of the compound represented by formula 1 has a thermogravimetric analysis pattern with a weight loss of 0.1066% from 25° C. to the melting point, where the “%” represents weight percentage;
(4) the crystal form III of the compound represented by formula 1 has a dynamic vapor sorption pattern with a moisture absorption weight gain of 0.4084% in the range of 0% to 95% relative humidity, where the “%” represents weight percentage.

3. The crystal form II or the crystal form III of the compound represented by formula 1 according to claim 2, wherein the X-ray powder diffraction pattern for the crystal form II of the compound represented by formula 1, using CuKα radiation and represented by 2θ angles, comprises characteristic peaks at 6.976°±0.2°, 8.928°±0.2°, 10.781°±0.2°, 12.775°±0.2°, 13.316°±0.2°, 13.967°±0.2°, 16.220°±0.2°, 16.450°±0.2°, 16.817°±0.2°, 17.598°±0.2°, 19.377°±0.2°, 19.494°±0.2°, 19.955°±0.2°, 20.875°±0.2°, 22.119°±0.2°, 24.798°±0.2°, 25.026°±0.2°, 25.752°±0.2°, 26.778°±0.2°, 27.959°±0.2°, 28.541°±0.2°, 28.957°±0.2°, 30.160°±0.2°, 33.201°±0.2°, 34.555°±0.2°, 34.916°±0.2°, 36.340°±0.2°, 36.602°±0.2°, and 37.986°±0.2°; and
the X-ray powder diffraction pattern for the crystal form III of the compound represented by formula 1, using CuKα radiation and represented by 2θ angles, comprises characteristic peaks at 5.515°±0.2°, 7.577°±0.2°, 8.137°±0.2°, 10.415°±0.2°, 11.632°±0.2°, 14.809°±0.2°, 16.258°±0.2°, 17.347°±0.2°, 19.318°±0.2°, 19.797°±0.2°, 20.813°±0.2°, 21.939°±0.2°, 22.663°±0.2°, 23.108°±0.2°, 24.664°±0.2°, 25.282°±0.2°, 25.998°±0.2°, 27.689°±0.2°, 28.387°±0.2°, 29.791°±0.2°, 30.347°±0.2°, 32.742°±0.2°, 34.932°±0.2°, 35.679°±0.2°, 37.573°±0.2°, and 38.218°±0.2°.

4. The crystal form II or the crystal form III of the compound represented by formula 1 according to claim 2, wherein the crystal form II satisfies one or more of the following conditions: (1) the X-ray powder diffraction pattern for the crystal form II of the compound represented by formula 1, using CuKα radiation and represented by 2θ angles, is substantially as shown in FIG. 5;
(2) the differential scanning calorimetry pattern for the crystal form II of the compound represented by formula 1 is substantially as shown in FIG. 6;
(3) the thermogravimetric analysis pattern for the crystal form II of the compound represented by formula 1 is substantially as shown in FIG. 7;
(4) the dynamic vapor sorption pattern for the crystal form II of the compound represented by formula 1 is substantially as shown in FIG. 8;
(5) infrared absorption spectrum for the crystal form II of the compound represented by formula 1, determined by KBr pellet method, is substantially as shown in FIG. 9; and
the crystal form III satisfies one or more of the following conditions:
(1) the X-ray powder diffraction pattern for the crystal form III of the compound represented by formula 1, using CuKα radiation and represented by 2θ angles, is substantially as shown in FIG. 10;
(2) the differential scanning calorimetry pattern for the crystal form III of the compound represented by formula 1 is substantially as shown in FIG. 11;
(3) the thermogravimetric analysis pattern for the crystal form III of the compound represented by formula 1 is substantially as shown in FIG. 12;
(4) the dynamic vapor sorption pattern for the crystal form III of the compound represented by formula 1 is substantially as shown in FIG. 13.

5-8. (canceled)

9. A preparation method for the crystal form II or the crystal form III of the compound represented by formula 1 according to claim 1, wherein the preparation method for the crystal form II is any one of the following schemes:
scheme 1:
the preparation method comprises the following steps: slurrying a crystal form I of the compound represented by formula 1 in acetonitrile, followed by drying to obtain the crystal form II of the compound represented by formula 1; wherein the crystal form I of the compound represented by formula 1 has an X-ray powder diffraction pattern using CuKα radiation and represented by 2θ angles comprising characteristic peaks at 8.086°±0.2°, 11.879°±0.2°, 14.375°±0.2°, 15.434°±0.2°, 16.213°±0.2°, 17.372°±0.2°, 17.618°±0.2°, 19.066°±0.2°, 19.897°±0.2°, 22.997°±0.2°, 23.240°±0.2°, 24.033°±0.2°, 25.339°±0.2°, 25.641°±0.2°, 30.179°±0.2°, 31.164°±0.2°, and 32.816°±0.2°;
scheme 2:
the preparation method comprises the following steps: mixing the compound represented by formula 1 with acetone, followed by heating, dissolving, cooling at a low temperature, and drying to obtain the crystal form II of the compound represented by formula 1;
scheme 3:
the preparation method comprises the following steps:
(1) mixing the compound represented by formula 1 with a good solvent, followed by heating and dissolving;
(2) mixing the solution from step (1) with an anti-solvent under stirring, followed by drying to obtain the crystal form II of the compound represented by formula 1; and
the preparation method for the crystal form III of the compound represented by formula 1 comprises the following steps:
(1) mixing a crystal form I of the compound represented by formula 1 with a mixed solvent of methanol/water;
(2) mixing the solution from step (1) with water under stirring;
(3) drying to obtain the crystal form III of the compound represented by formula 1;
wherein the crystal form I of the compound represented by formula 1 has an X-ray powder diffraction pattern using CuKα radiation and represented by 2θ angles comprising characteristic peaks at 8.086°±0.2°, 11.879°±0.2°, 14.375°±0.2°, 15.434°±0.2°, 16.213°±0.2°, 17.372°±0.2°, 17.618°±0.2°, 19.066°±0.2°, 19.897°±0.2°, 22.997°±0.2°, 23.240°±0.2°, 24.033°±0.2°, 25.339°±0.2°, 25.641°±0.2°, 30.179°±0.2°, 31.164°±0.2°, and 32.816°±0.2°.

10. The preparation method for the crystal form II or the crystal form III of the compound represented by formula 1 according to claim 9, wherein the preparation method for the crystal form II of the compound represented by formula 1 satisfies one or more of the following conditions:
(1) in scheme 1, the volume-to-mass ratio of the acetonitrile to the compound represented by formula 1 is 10 to 30 times;
(2) in scheme 1, the slurrying is further followed by rotating at room temperature for equilibration;
(3) in scheme 1, the drying is natural drying;
(4) in scheme 2, the mixing is carried out at a rotational speed of 400 rpm;
(5) in scheme 2, the volume-to-mass ratio of the acetone to the compound represented by formula 1 is 20 to 40 times;
(6) in scheme 2, the heating is carried out in a water bath at a temperature of 40° C. to 60° C.;
(7) in scheme 2, the dissolving is further followed by filtrating;
(8) in scheme 2, the cooling is carried out at a temperature of −10° C. to −30° C. or less;
(9) in scheme 2, the cooling is carried out for 10 hours to 16 hours;
(10) in scheme 2, the drying is natural drying;
(11) in scheme 3, the good solvent is one of ethyl acetate, acetone, dichloromethane, or tetrahydrofuran;
(12) in scheme 3, the heating is carried out in a water bath at a temperature of 40° C. to 60° C.;
(13) in scheme 3, the anti-solvent is n-heptane or methyl tert-butyl ether;
(14) in scheme 3, the volume-to-mass ratio of the good solvent to the compound represented by formula 1 is 30 to 180 times;
(15) in scheme 3, the volume ratio of the good solvent to the anti-solvent is 1:(0.5 to 5);
(16) in scheme 3, in step (2), the mixing is carried out by dropwise addition of the anti-solvent to the solution from step (1); and
(17) in scheme 3, the drying is natural drying; and
the preparation method for the crystal form III of the compound represented by formula 1 satisfies one or more of the following conditions:
(1) in the step (1), the volume ratio of the methanol to the water is (5 to 15):1;
(2) in the step (1), the volume-to-mass ratio of the mixed solvent to the compound represented by formula 1 is 90 to 110 times;
(3) in the step (2), the volume-to-mass ratio of the water to the compound represented by formula 1 is 20 to 30 times;
(4) in the step (2), the mixing is carried out by dropwise addition of water to the solution from step (1);
(5) in the step (2), the mixing is carried out at a temperature of 45° C. to 55° C.;
(6) in the step (3), the drying is vacuum drying; and
(7) in the step (3), the drying is carried out at a temperature of 40° C. to 50° C.

11-12. (canceled)

13. A pharmaceutical composition comprising a therapeutically effective amount of one or two of the crystal form II and the crystal form III of the compound represented by formula 1 according to claim 1 and a pharmaceutically acceptable excipient.

14. A JAK kinase inhibitor comprising the crystal form II or the crystal form III of the compound represented by formula 1 according to claim 1.

15. A method for treating a disease related to JAK kinase, cancer or an immune disease in a subject in need thereof, comprising administering an effective amount of the crystal form II or the crystal form III of the compound represented by formula 1 according to claim 1.

16. (canceled)

17. The preparation method for the crystal form II or the crystal form III of the compound represented by formula 1 according to claim 10, wherein the preparation method for the crystal form II of the compound represented by formula 1 satisfies one or more of the following conditions:
(1) in scheme 1, the volume-to-mass ratio of the acetonitrile to the compound represented by formula 1 is 20 times;
(2) in scheme 2, the volume-to-mass ratio of the acetone to the compound represented by formula 1 is 30 times;
(3) in scheme 2, the heating is carried out in a water bath at a temperature of 50° C.;
(4) in scheme 2, the cooling is carried out at a temperature of −20° C.;
(5) in scheme 2, the cooling is carried out for 12 hours;
(6) in scheme 3, the heating is carried out in a water bath at a temperature of 50° C.;
(7) in scheme 3, the volume-to-mass ratio of the good solvent to the compound represented by formula 1 is 30 times, 50 times, 90 times, or 180 times; and
(8) in scheme 3, the volume ratio of the good solvent to the anti-solvent is 1:0.8, 1:1, 1:1.7, 1:2.9, 1:3.2, or 1:4.2; and
the preparation method for the crystal form III of the compound represented by formula 1 satisfies one or more of the following conditions:
(1) in the step (1), the volume ratio of the methanol to the water is 10:1;
(2) in the step (1), the volume-to-mass ratio of the mixed solvent to the compound represented by formula 1 is 100 times;
(3) in the step (2), the volume-to-mass ratio of the water to the compound represented by formula 1 is 25 times;
(4) in the step (2), the mixing is carried out at a temperature of 50° C.;
(5) in the step (3), the drying is carried out at a temperature of 45° C.

18. The pharmaceutical composition according to claim 13, the pharmaceutical composition is in the form of a tablet, a pill, a powder, a solution, an emulsion, an ointment, a capsule, or a liniment.

19. A JAK kinase inhibitor comprising the pharmaceutical composition according to claim 13.

20. The method according to claim 15, wherein the crystal form II or the crystal form III of the compound represented by formula 1 is used in combination with other therapeutic agents for treating a disease related to JAK kinase, cancer or an immune disease.

21. The method according to claim 20, wherein the other therapeutic agents are used for treating a disease related to JAK kinase, cancer or an immune disease.

22. The method according to claim 15, wherein the cancer is one or more of myeloproliferative neoplasm, lymphoma, and leukemia; and
the immune disease is one or more of rheumatoid arthritis, alopecia areata, atopic dermatitis, vitiligo, and psoriasis.

23. A method for treating a disease related to JAK kinase, cancer or an immune disease in a subject in need thereof, comprising administering an effective amount of the pharmaceutical composition according to claim 13.

24. The method according to claim 23, wherein the pharmaceutical composition is used in combination with other therapeutic agents for treating a disease related to JAK kinase, cancer or an immune disease.

25. The method according to claim 24, wherein the other therapeutic agents are used for treating a disease related to JAK kinase, cancer or an immune disease.

26. The method according to claim 23, wherein the cancer is one or more of myeloproliferative neoplasm, lymphoma, and leukemia; and
the immune disease is one or more of rheumatoid arthritis, alopecia areata, atopic dermatitis, vitiligo, and psoriasis.
